(19) United States
(12) Patent Application Publication
(10) Pub. No.: US 2007/0051826 A1
(43) Pub. Date: Mar. 8, 2007

(54) Air freshener and combination container and lid

(76) Inventor: Joshua Schofield, Laconia, NH (US)

Correspondence Address: Lawson & Persson, P.C., Suite 103, 67 Water Street, Laconia, NH, 03246, US

(21) Appl. No.: 11/208,503

(22) Filed: Aug. 22, 2005

(51) Int. Cl. A61L 9/04

(52) U.S. Cl. 239/60; 239/57; 239/34

(57) ABSTRACT

An air freshener and combination container and lid for forming an air freshener. The air freshener includes a scented wax solid, a container, a lid, and an attachment for attaching the container to a surface. The container is manufactured of a thermally conductive material and has a bottom and at least one sidewall defining an interior, an exterior, and an open top. The interior of the container is dimensioned such that scented wax solid fits within the interior. The lid is removably attached to the open top of the container and includes a seal and a plurality of vent openings disposed therethrough. The air freshener prevents liquid wax from escaping through the vent openings when the container is moved in a direction parallel to the bottom of the container.

### FIELD OF THE INVENTION

The present invention relates to the field of air fresheners and, in particular, to air fresheners and air freshener kits that disperse a scent from a scented medium disposed Within a container.

### BACKGROUND OF THE INVENTION

It is common for unpleasant odors to develop within motor vehicles. For example, if the owner of the vehicle is a smoker, the smell of tobacco can permeate the upholstery and leave behind an unpleasant odor, which may remain even after the vehicle has been parked for an extended period of time. If the vehicle owner leaves a window open, musty smells may develop. If food or drinks are spilled, the residue thereof may likewise produce an unpleasant odor. As these examples show, there are a number of reasons why unpleasant odors may develop within a motor vehicle.

Regardless of how odors develop, it is common for owners of vehicles to use air fresheners to mask these odors. One common type of air freshener is manufactured of paper that is impregnated with a scented volatilizable substance. The paper is then sealed within an airtight plastic package, which prevents the scent from dispersing until the package is opened. These air fresheners come in many shapes; the most of common of which are “Christmas Tree” shaped, and come in a limited number of scents. Unfortunately, these air fresheners have a number of drawbacks. First, they are relatively short lived; lasting no more then two weeks before they must be replaced. Second, they are prone to staining surfaces to which they come in contact and, consequently, must hang freely. Because of this requirement, they often are hung from the vehicle's rear view mirror, which is distracting to the driver. Third, because of their short life span, the scent is extremely strong when first opened and rapidly becomes very weak. Finally, although different scents are available, there are not a wide variety of scents and those that are available are undesirable to many people.

U.S. Pat. No. 4,306,679 discloses a dispenser that houses a similar paper air freshener impregnated with a scented volatilizable substance and allows the release of its scent to be controlled by controlling the size of the opening through which air may pass into the container. This dispenser of this patent is helpful in eliminating the possibility of staining and reducing the strength of the scent when first opened. However, these dispensers do not significantly extend the life of the air freshener. Further, they are not adapted for reuse, making them more expensive to use over time. Finally, they suffer from the same limitations on the number of available scents.

U.S. Pat. No. 5,478,505 disclosed another device directed to freshening air within a vehicle. The device of this patent discloses a container that is attached to a vehicle's air vent and disperses scent by forcing air through a membrane impregnated with a scented volatilizable substance. As was the case with the dispenser of U.S. Pat. No. 4,306,679, this device is helpful in eliminating the possibility of staining and allows the strength of the scent to be controlled after it is first opened. However, these dispensers do not significantly extend the life of the air freshener and are likewise not adapted for reuse, making them more expensive to use over time. Further, these air fresheners are available in a more limited number of scents than those described above.

Still another common type of air freshener is a gel type air freshener. These air fresheners utilize a fragrant gel, which is dispersed from a container disposed in a cup-holder or other interior holding device. Gel air fresheners are longer lasting than the scented paper or membrane type air fresheners and reduce the chance of staining the interior of the vehicle. However, the number of available scents is very limited, and they still last no more than a month in use and are typically not adapted for reuse, again making them more expensive to use over time.

Still another common type of auto air freshener is a liquid air freshener that may attach to an air vent or be held in a vehicle's cup holder. These air fresheners work through evaporation of the liquid and have a significantly longer life than the paper and membrane based fresheners discussed above. Further, many of these air fresheners may be refilled after the liquid is exhausted, reducing the overall cost of use over time. However, these air fresheners are also limited by the number of scents available and are more difficult to store and handle due to the nature of the liquid; i.e. it is prone to spillage and breakage due to freezing.

Therefore, there is a need for an air freshener that is adapted for use within a vehicle, will lasting more then one month before it must be replaced, is not prone to staining surfaces to which they come in contact, does not need to be mounted in a location that is distracting to the driver, does not have an extremely strong scent when first opened and rapidly becomes very weak thereafter, is readily reusable, uses commonly available freshening media, and which are available in a large number of scents.

### SUMMARY OF THE INVENTION

The inventor developed the air freshener of the present invention in response to a trip to a “Yankee Candle” retail store. During his visit to the store, he smelled a candle with a scent of hot buttered rum and immediately wanted to experience it in his vehicle. Consequently, he bought a package of scented wax tarts and set about to develop out a way to use them in his vehicle.

The first issue that he identified during the design process was finding a container that would conduct heat to allow the wax to melt into a liquid, which better disperses its scent, and would be of a proper size to hold the wax tart. He found that a round tin of ALTOIDS brand mints, which presents as a short cylindrical container, was properly sized to fit the wax tart and, being made of metal, was thermally conductive. Accordingly, he adhered two strips of hook and loop fastener to the bottom of the tin, disposed the tart within the container, attached the lid to the tin, and affixed the tin to the dashboard of his vehicle. He then removed the lid from the tin when he entered the vehicle, in order to begin experiencing the scent, and reattached the lid when the vehicle was parked.

This first prototype worked better than he had imagined. The inventor found that mounting the container on the dashboard provided sufficient heating of the container to melt the wax tart, even in the cold winter months. Further, the scent was extremely uniform from the time that it was first installed until the tart was finally spend approximately six months thereafter. The one drawback that was identified was the tendency for the liquefied wax to spill over the sides of the open top tin while driving.

In response to this problem, the inventor disposed a series of holes through the lid of the tin, which acted as vent openings and allowed the lid to be attached to the container at all times. This solution worked reasonable well, although the inventor did experience flow through the outermost holes in the tin when the vehicle went around sharp corners. Accordingly, the inventor sought to develop ways of modifying the lid to allow the scent to disperse, while preventing spillage of the liquefied wax.

After experimenting with a number of solutions, the inventor developed two specific ways to prevent the escape of the liquid wax from the vent openings. The first was a modification of the hole pattern in the lid to concentrate the openings in the center, thus preventing spillage of the wax. This worked reasonably well, and worked extremely well in circumstances where the height of the combined container and lid were increased. Thus, the first such solution involves dimensioning the height of the container and the disposal of the holes relative to the sidewall such that the liquid wax would not escape during normal use.

The second solution involved the disposal of an absorbent material on the inside surface of the lid in the area where the holes were disposed. The absorbent material chosen was an open cell foam material, which would absorb any of the liquid wax that came into contact therewith, while still allowing air to flow therethrough to cause a dispersion of the scent. This solution worked even better than the dimensioning of the container and holes and, therefore, is preferred.

The inventor envisions the combination container and lid being sold as stand alone units for combination with existing wax solids, and as complete air fresheners that include wax solids. As noted above, the sale as stand alone units will allow a wide variety of different scented waxes to be utilized and, therefore, this is currently preferred. However, it may be that a candle manufacturing company, such as Yankee Candle, would sell these air fresheners for single use as a full combination. Accordingly, the present invention is intended to include such a combination.

The present invention is an air freshener and combination container and lid for forming an air freshener that embodies the design described above and overcome the drawbacks of conventional air fresheners. The air freshener includes a scented wax solid, a container, a lid and a means for attaching the container to a surface. The wax solid has a first surface, a second surface and a thickness defining an outer edge. The container is manufactured of a thermally conductive material and has a bottom and at least one sidewall defining an interior, an exterior, and an open top. The sidewall of the container has a substantially uniform height, which is greater than the thickness of the scented wax solid. The interior of the container is dimensioned such that the outer edge of the scented wax solid fits within the interior. The lid is dimensioned for removable attachment to the open top of the container and includes a plurality of vent openings disposed therethrough. The air freshener also includes a sealing means for attaching the lid to the container such that a liquid wax may not escape from the container, and also means for preventing the liquid wax from escaping through the vent openings when the container is moved in a direction parallel to the bottom of the container.

In its assembled form, the scented wax solid, preferably a wax tart, is disposed within the interior of the container, the lid is attached to the open top, and a means for attaching the bottom of the container to a surface is attached to the bottom of the exterior of the container. In operation, the scented wax solid is heated by the bottom and sidewall of the container and becomes a liquid wax, which emits a scent that passes through the vent openings and delivers the scent to the area surrounding the air freshener. As noted above, the preferred means for preventing the liquid wax from flowing through the vent openings When the container is moved in a direction parallel to the bottom of the container is a sheet of an absorbent material disposed upon the interior of the lid and dimensioned to cover said vent openings. This absorbent material is preferably a sheet of an open cell foam, but may any material capable of absorbing the liquid wax while still allowing dispersion of the wax scent therethrough.

In other embodiments, the container and lid are dimensioned such that a liquid wax may not escape through the vent openings when said container is moved in a direction parallel to the bottom of the container. This is preferably accomplished by dimensioning the container and lid, and disposing the vent openings through the lid, in such manner as to prevent the wax from escaping. This may entail making the sidewalls of the container longer, making the sidewalls of the lid longer, and/or moving the vent openings further inward from the sidewalls of the lid. In such embodiments using a standard said wax tart having a thickness of between 0.25 and 0.75 inches and a maximum circumference of 3 inches, the distance between the bottom of the container and the top of the lid is between 0.8 inches and 1.0 inches, and the vent openings in the lid are disposed at least 0.75 inches from the sidewall. However, other variations would achieve similar results and, therefore, the invention should not be seen as being so limited.

In the preferred embodiment, the container is manufactured of metal and the sealing means for attaching the lid to the container such that a liquid wax may not escape from the container is a substantially semicircular mating ridge extending from the exterior of the sidewall of the container proximate the top of the container and a substantially semicircular groove extending inward into the interior of the sidewall of the lid. However, containers and lids having mating threads or other art recognized sealing means may be substituted to achieve similar results.

The preferred means for attaching the bottom of the container to a surface is an adhesive backed hook and loop fastener. However, other means, such as double sided tape, sheets of non-skid material, or the like, may be substituted and are contemplated herein.

Therefore, it is an aspect of the invention to provide an air freshener that is adapted for use within a vehicle.

It is a further aspect of the invention to provide an air freshener that will last more then one month before it must be replaced.

It is a further aspect of the invention to provide an air freshener that is not prone to staining surfaces to which they come in contact.

It is a further aspect of the invention to provide an air freshener that does not need to be mounted in a location that is distracting to the driver.

It is a further aspect of the invention to provide an air freshener that does not have an extremely strong when first opened and rapidly becomes very weak thereafter.

It is a further aspect of the invention to provide an air freshener that is readily reusable.

It is a further aspect of the invention to provide an air freshener that uses commonly available freshening media available in a large number of scents.

It is a still further aspect of the invention to provide an air freshener that may be sold in kit form including a container and at least one scented wax tart.

These aspects of the invention are not meant to be exclusive and other features, aspects, and advantages of the present invention will be readily apparent to those of ordinary skill in the art when read in conjunction with the following description, appended claims and accompanying drawings.

### BRIEF DESCRIPTION OF THE DRAWINGS

FIG. 1 is a cut away side view of the preferred embodiment of the air freshener of the present invention.

FIG. 2 is a cut away side view of an alternative embodiment of the air freshener of the present invention.

FIG. 3 is a cut away side view of the embodiment of the air freshener of FIG. 2 showing the dimensional relationships between the various parts of the air freshener.

FIG. 4 is a top view of one embodiment of the lid of the air freshener of FIGS. 2 and 3 showing a preferred arrangement of vent openings therethrough.

### DETAILED DESCRIPTION OF THE INVENTION

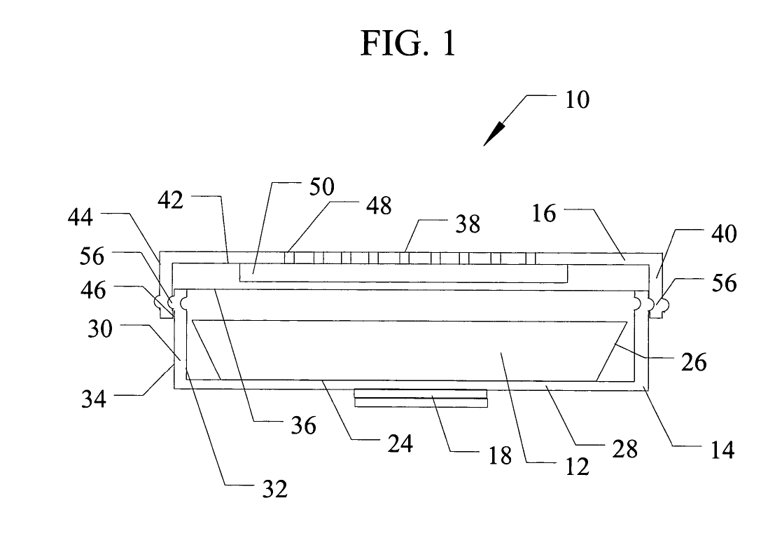

Referring first to FIG. 1, the preferred air freshener 10 includes a scented wax solid 12, a container 14, a lid 16, and a hook and loop fastener 18 for attaching the container 14 to a surface.

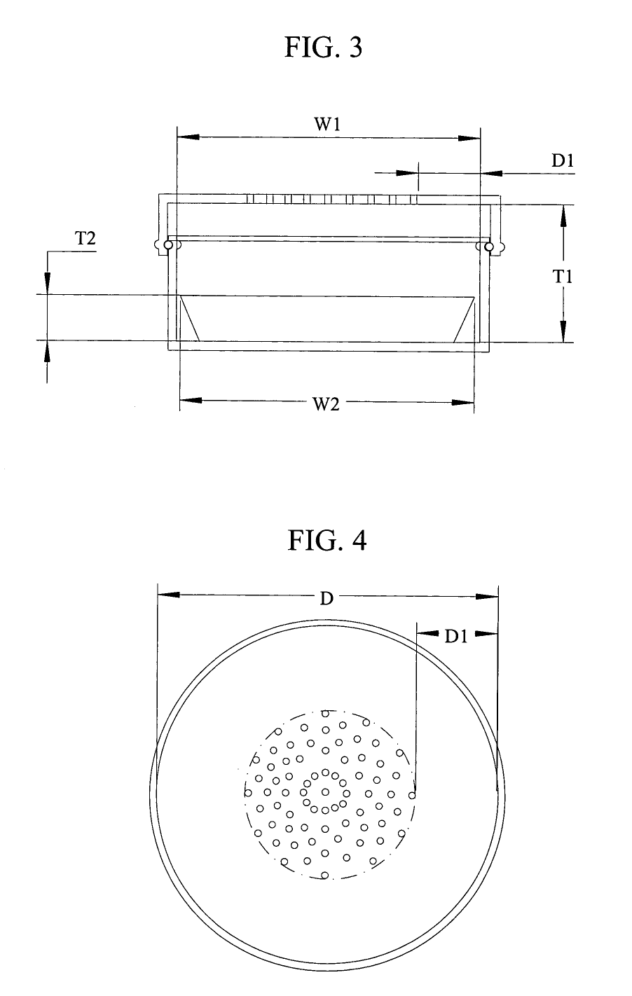

The wax solid 12 is preferably a scented wax commonly referred to as a “wax tart”. The wax solid 12 has a top surface 22, a bottom surface 24 and a thickness (denoted as T2 in FIG. 3) defining an outer edge 26. The preferred wax solid 12 is a tart shaped scented wax solid of the type sold by the Yankee Candle Company of South Deerfield, Mass., under the trademark TARTS® Wax Potpourri. The preferred wax solid 12 presents as a conic section, with the top surface 22 being of larger diameter than the bottom surface, and has a substantially scalloped edge. However, wax solids may come in a variety of shapes, including those that present as cylindrical sections, prisms or the like. The wax solid 12 is preferably a low melting point paraffin wax, which melts at a temperature of approximately 125° Fahrenheit, as these waxes are easily melted even in winter. However, wax solids having higher melting temperatures may be utilized in regions in which the combination of ambient temperature and solar heating are such that the temperature of the container 14 will exceed 125° Fahrenheit.

The container 14 is manufactured of a thermally conductive material, preferably metal, and has a bottom and at least one sidewall 30 defining an interior surface 32, an exterior surface 34, and an open top 36. The sidewall 30 of the container 14 has a substantially uniform height (denoted as H2 in FIG. 3), which is greater than the thickness of the scented wax solid 12. The interior surface 32 of the container 14 is dimensioned such that the entire outer edge 26 of the scented wax solid 12 fits within the interior surface 32. As shown in FIG. 1, the space between the outer edge 26 of the scented wax solid 12 and the interior surface 32 proximate to the sidewall 30 is minimal. However, the container 12 may be dimensioned to create large spaces, or to snugly fit the scented wax solid 12 therein.

The lid 16 is dimensioned for removable attachment to the open top 36 of the container 12 and includes a top 38, and a sidewall 40, which define an interior surface 42, and exterior surface 44 and an open bottom 46. The lid 16 is preferably manufactured of the same thermally conductive material as the container 14 so that liquid wax coming in contact therewith will quickly melt and flow back onto the container 14. However, the lid 16 may be manufactured of different materials, such as plastics, natural or synthetic rubbers, or other non-conductive materials, to achieve similar, albeit slightly inferior, results.

A plurality of vent openings 48 are disposed through the top 38 of the lid 16 to provide airflow into and out of the container 14. The vent openings 48 are preferably cylindrical holes that are disposed in a pattern along the top 38 of the lid 16. The size and number of such vent openings 48 is not of primary importance. However, as described with reference to FIGS. 3 and 4, the vent openings 48 are preferably disposed a substantial distance from the sidewall 40 of the lid 16 to prevent the escape of liquid wax during normal operation. Notwithstanding the preferability of this arrangement, in those embodiments that include a sheet 50 of absorbent material, such as the embodiment of FIG. 1, the vent openings 48 may extend all the way to the sidewall 40 without any degradation in performance.

In the embodiment of FIG. 1, the container 12 includes a substantially semicircular ridge 54 extending from said exterior surface 34 of the sidewall 30 proximate to the open top 36 of said container and the lid include a substantially semicircular groove 56 extending inward into said interior surface 40 of the sidewall 40 of the lid 16. The groove 56 of the lid 16 is dimensioned to mate with the ridge 54 of the container 14 such that, when the lid is attached to the container, the liquefied wax may not escape from the container 14. However, in other embodiments utilizing rigid materials for the lid 16 and container 14, the ridge 54 and groove 56 are replaced by mating threaded portions (not shown) on the interior surface 40 of the lid 16 and the exterior surface 34 of the container 12. In other embodiments, the container 12 includes a groove (not shown) in which an O-ring is disposed. In these embodiments, the O-ring and the interior surface 40 of the sidewall 40 of the lid 16 are dimensioned such that the sidewall 40 compresses the O-ring, effectively holding the lid 16 in place and sealing the lid 16 and container. In embodiments in which the lid 16 is manufactured from an elastic material, such as plastic or rubber, the container may include a lip (not shown) about the open top 36 and the sidewalls 40 of the lid 16 are dimensioned to expand over and compress against the lip, in a manner similar to those used for resealable foodstuffs, such as canned nuts. Regardless of which arrangement is chosen, however, all embodiments of the invention include some sealing means for attaching the lid 16 to the container 14 such that a liquified wax may not escape from the container 16.

The embodiment of FIG. 1 includes a sheet 50 of an absorbent material, which prevents the liquified wax from escaping through the vent openings 48 when the container 14 is moved in a direction parallel to the bottom 28 of the container 16. The preferred sheet 50 of absorbent material is manufactured from an open cell foam material, but may be any material capable of absorbing the liquified wax while still allowing dispersion of the Wax scent therethrough.

As noted above, a hook and loop fastener 18 is disposed on the bottom 28 of the exterior surface 34 of the container 14 and is used to removably attach the container 14 to a surface 62 of the vehicle; preferably the surface of the dashboard proximate to the vehicle's hot air vents. The hook and loop fastener 18 preferably includes an adhesive (not shown) attached to the bottom thereof to allow one portion of the fastener 18 to stick directly to the dashboard As shown in FIG. 1, the hook and loop fastener 18 is disposed in proximate to the center of the bottom 28 of the container 14. However, it is recognized that multiple strips of hook and loop fastener 18 may be attached at various points along the bottom 28 of the container 14. In other embodiments, the hook and loop fastener 18 is replaced by double sided adhesive for adhering the container 14 directly to the dashboard. This adhesive may be a conventional double-sided adhesive, providing a permanent attachment, or may be a removable adhesive. In other embodiments, rubber feet or other non-slip materials may be attached to the bottom 28 of the container 14 to provide a frictional engagement with the surface 62. In still others, a mechanical closure, such as a snap, is attached to the container and a mating closure is adhered to the surface 62. As may be seen from the foregoing examples, any number of art recognized means may be employed to attach the container 14 to the surface 62 and, therefore, the invention should not be seen as being limited to those described including those exemplary means described herein.

In its assembled form, the scented wax solid 12 is disposed upon the bottom 24 of the interior surface 32 of the container 14. The lid 16 is attached to the open top 36 of the container 16, and the bottom 28 of the container 14 is attached to a surface of the vehicle. In the winter months, the exterior surface 34 of the container 14 is heated primarily by conduction and convection from the surface 62 of the vehicle proximate to the defrost vents, with solar heating through the windshield providing secondary heating. However, in summer months, solar heating through the windshield providing primary heating, with heating from the surface 62 being secondary. To enhance the solar heating of the container 14 and lid 16, it is preferred that both the container 14 and lid 16 have exterior surfaces 34, 44 that absorb rather than reflect light. Accordingly, it is preferred that the exterior surfaces 34, 44 be finished in dark colors. Regardless of its source, the heat conducts through the bottom 28 and the sidewall 30 of the container 14, at least one of which is in communication with the scented wax solid 12. The heat then conducts into the wax solid 12, which consequently changes phase to a liquefied wax, from which the scent is evaporated into the air within the container. The evaporated scent then passes through the vent openings 48 and delivers the scent to the area surrounding the air freshener 10.

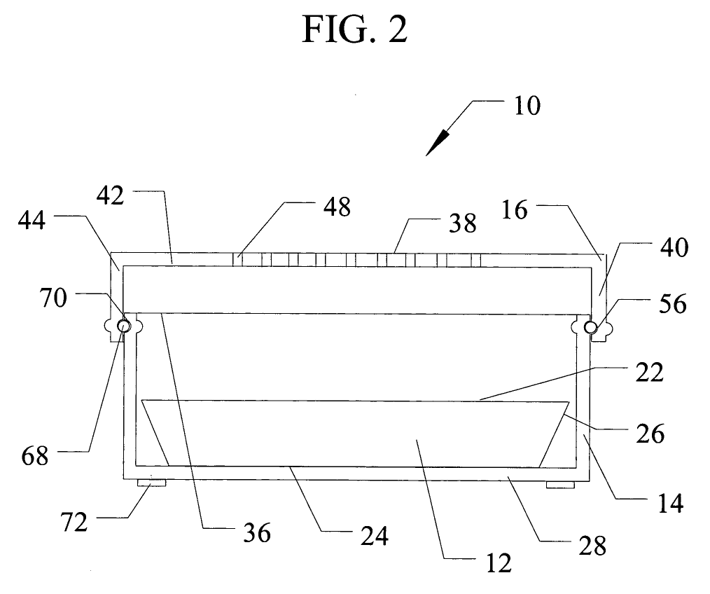

Referring now to FIG. 2, an alternative embodiment of the air freshener 10 of the present invention is shown. The embodiment of FIG. 2 is similar in most respects to the embodiment of FIG. 1. It includes a container 14 having a bottom 28 and a side wall 30 defining an interior surface 32 and an exterior surface 34. The air freshener 10 also includes a lid 16 having a plurality of vent openings 48 through its top 38. The air freshener 10 of the embodiment of FIG. 2 differs from the air freshener of the embodiment of FIG. 1 in three respects.

First, the embodiment of FIG. 2 does not include the absorbent material 50 of the embodiment of FIG. 1 as its means for preventing the liquid wax from flowing through the vent openings 48 when said container 14 is moved in a direction parallel to its bottom 28 of said container. Rather, the air freshener 10 of FIG. 2 utilizes the dimensional characteristics of the combination container 14 and lid 16, and the disposition of the vent openings 48 toward the center of the lid 16 to prevent the liquid wax from flowing through the vent openings 48.

The dimensional characteristics of the air freshener 10 of FIG. 2 are described with reference to FIGS. 3 and 4. As shown in FIG. 3, the wax solid 12 has a width W2 and a thickness T2. The interior surface of the container as a width W1 and a height H2. The combination container 14 and lid 16 has an interior height T1, which extends from the bottom 28 of the container 14 to the interior surface 42 of the top 48 of the lid 16. The vent openings 48 in the lid 16 are disposed a distance D1 from the sidewall 40 of the lid 16. It has been found that, in embodiments not utilizing an absorbent material 50, dimensioning the interior height T1 to be at least 150% of the thickness of the wax solid 12 and the distance D1 to be at least 20% of a diameter D of the lid prevents the liquid wax from flowing through the vent openings 48. For example, in embodiments utilizing typical scented wax solids having thicknesses between 0.5 and 1.0 inches and widths of between 2 inches and 3 inches, the interior height T1 is between 0.75 inches and 2.5 inches, the diameter D of the lid is between 2.5 inches and 3.5 inches, and the distance D1 is at least 0.5 inches. In the preferred embodiment, which utilizes a wax tart sold by the Yankee Candle Company, the width W of the wax solid 12 is approximately 2.3 inches and its thickness T2 is approximately 0.7 inches. In such embodiments, is preferred that the height H2 of the container 14 be approximately one inch, the width W1 of the container be approximately 3.15 inches, the interior height T1 be approximately 1.2 inches, the diameter of the lid D be 3.2 inches and the distance D1 be at least 0.64 inches.

Referring again to FIG. 2, the other difference between the embodiments of FIG. 2 and FIG. 1 are the sealing means and the attachment means utilized in the air freshener 10 of FIG. 2. The air freshener 10 of FIG. 2 utilizes an O-ring arrangement in which an O-ring 68 is disposed within a groove 70 disposed about the circumference of the container 14. The O-ring 68 mates with the groove 56 disposed within the inside of the side wall 40 of lid 16 to seal the lid 14 and the container 16 together. The air freshener 10 of FIG. 2 also substitutes rubber feet 72 for the hook and loop fastener 18 of FIG. 1. As noted above, a wide variety of sealing means and attachment means may be utilized, and any of the sealing means or attachment means discussed above may be substituted for those shown in FIG. 2 to achieve similar results.

Although the present invention has been described in considerable detail with reference to certain preferred versions thereof, other versions would be readily apparent to those of ordinary skill in the art. Therefore, the spirit and scope of the appended claims should not be limited to the description of the preferred versions contained herein.

What is claimed is:

1. An air freshener comprising:
a scented wax solid having a first surface, a second surface and a thickness defining an outer edge;
a container having a bottom and at least one side wall defining an interior surface and an exterior surface, and an open top, wherein said at least one side wall of said container has a substantially uniform height, said height being greater than said thickness of said scented wax solid, wherein said interior of said container is dimensioned such that said outer edge of said scented wax solid fits within said interior, and wherein said container is manufactured of a thermally conductive material;
a lid dimensioned for removable attachment to said open top of said container, said lid comprising a sidewall and a top defining an interior surface and an exterior surface, wherein said top of said lid comprises a plurality of vent openings disposed therethrough;
sealing means for attaching said lid to said container such that a liquid wax may not escape from a junction formed by said lid and said container;
means for attaching said bottom of said container to a surface; and
means for preventing the liquid wax from flowing through said vent openings in said lid when said container is moved in a direction parallel to said bottom of said container;
wherein said scented wax solid is disposed within said interior of said container, said lid is attached to said open top, and said means for attaching said bottom of said container to a surface is attached to said bottom of said exterior of said container.

2. The air freshener as claimed in claim 1 wherein said scented wax solid is a wax tart.

3. The air freshener as claimed in claim 2 wherein said means for preventing the liquid wax from flowing through said vent openings when said container is moved in a direction parallel to said bottom of said container comprises a sheet of an absorbent material disposed upon said interior of said lid and dimensioned to cover said vent openings.

4. The air freshener as claimed in claim 5 wherein said absorbent material is an open cell foam material.

5. The air freshener as claimed in claim 1 wherein said container is manufactured of metal.

6. The air freshener as claimed in claim 1 wherein said sealing means for attaching said lid to said container such that a liquid wax may not escape from said container comprises a substantially semicircular ridge extending from said exterior of said sidewall of said container proximate said top of said container and a substantially semicircular groove extending inward into said interior of said sidewall of said lid, said groove being dimensioned to mate with said ridge.

7. The air freshener as claimed in claim 1 wherein said means for attaching said bottom of said container to a surface comprises an adhesive backed hook and loop fastener.

8. An air freshener comprising:
a scented wax solid having a first surface, a second surface and a thickness defining an outer edge;
a container having a bottom and at least one side wall defining an interior surface and an exterior surface, and an open top, wherein said at least one side wall of said container has a substantially uniform height, said height being greater than said thickness of said scented wax solid, wherein said interior of said container is dimensioned such that said outer edge of said scented wax solid fits within said interior, and wherein said container is manufactured of a thermally conductive material;
a lid dimensioned for removable attachment to said open top of said container, said lid comprising a sidewall and a top defining an interior surface and an exterior surface, wherein said top of said lid comprises a plurality of vent openings disposed therethrough;
sealing means for attaching said lid to said container such that a liquid wax may not escape from a junction formed by said lid and said container; and
means for attaching said bottom of said container to a surface;
wherein said container and said lid are dimensioned such that a liquid wax may not escape through said vent openings when said container is moved in a direction parallel to said bottom of said container; and
wherein said scented wax solid is disposed within said interior of said container, said lid is attached to said open top, and said means for attaching said bottom of said container to a surface is attached to said bottom of said exterior of said container.

9. The air freshener as claimed in claim 8 wherein a distance between said bottom of said interior of said container and said interior of said top of said lid and a location of said vent openings through said lid are dimensioned and disposed such that said liquid wax may not escape through said vent openings when said container is moved in the direction parallel to said bottom of said container.

10. The air freshener as claimed in claim 9 wherein said distance between said bottom of said container and said top of said lid is at least 150% of said thickness of said wax solid and wherein said vent openings are disposed a distance from the sidewall of the lid that is at least 20% of a diameter of said lid.

11. The air freshener as claimed in claim 10 wherein said scented wax solid is a wax tart having thickness between 0.5 and 1.0 inches and a width of between 2 inches and 3 inches, wherein said distance between said bottom of said container and said top of said lid is between 0.75 inches and 2.5 inches, wherein said diameter of said lid is between 2.5 inches and 3.5 inches, and wherein said vent openings in said lid are disposed at least 0.5 inches from said at least one sidewall of said lid.

12. The air freshener as claimed in claim 8 further comprising a sheet of an absorbent material disposed upon said interior of said lid and dimensioned to cover said vent openings.

13. The air freshener as claimed in claim 12 wherein said absorbent material is an open cell foam material.

14. The air freshener as claimed in claim 8 wherein said container is manufactured of metal.

15. The air freshener as claimed in claim 8 wherein said sealing means for attaching said lid to said container such that a liquid wax may not escape from said container comprises a substantially semicircular ridge extending from said exterior of said sidewall of said container proximate said top of said container and a substantially semicircular groove extending inward into said interior of said sidewall of said lid, said groove being dimensioned to mate with said ridge.

16. The air freshener as claimed in claim 1 wherein said means for attaching said bottom of said container to a surface comprises an adhesive backed hook and loop fastener.

17. A combination container and lid for holding a scented wax solid to form an air freshener, said combination comprising:
a container comprising a bottom and at least one side wall defining an interior surface and an exterior surface, and an open top, wherein said at least one side wall of said container has a substantially uniform height, said height being greater than a thickness of said scented wax solid, wherein said interior of said container is dimensioned such that an outer edge of said scented wax solid fits within said interior, and wherein said container is manufactured of a thermally conductive material;
a lid dimensioned for removable attachment to said open top of said container, said lid comprising a sidewall and a top defining an interior surface and an exterior surface, wherein said top of said lid comprises a plurality of vent openings disposed therethrough;
sealing means for attaching said lid to said container such that a liquid wax may not escape from a junction formed by said lid and said container;
means for attaching said bottom of said container to a surface; and
means for preventing the liquid wax from flowing through said vent openings in said lid when said container is moved in a direction parallel to said bottom of said container.

18. The combination container and lid as claimed in claim 17 wherein said means for preventing the liquid wax from flowing through said vent openings when said container is moved in a direction parallel to said bottom of said container comprises a sheet of an absorbent material disposed upon said interior of said lid and dimensioned to cover said vent openings.

19. The combination container and lid as claimed in claim 18 wherein said absorbent material is an open cell foam material.

20. The combination container and lid as claimed in claim 17 wherein said container and said lid are manufactured of metal.
